(19) United States
(12) Patent Application Publication
(10) Pub. No.: US 2020/0245860 A1
(43) Pub. Date: Aug. 6, 2020

(54) OPHTHALMOLOGIC APPARATUS

(71) Applicant: NIDEK CO., LTD., Gamagori (JP)

(72) Inventors: Hiroyuki UMANO, Gamagori (JP); Kenji AOKI, Gamagori (JP); Yusuke OOGI, Gamagori (JP)

(73) Assignee: NIDEK CO., LTD., Gamagori (JP)

(21) Appl. No.: 16/779,850

(22) Filed: Feb. 3, 2020

(30) Foreign Application Priority Data
Feb. 4, 2019 (JP) 2019-018248
Feb. 4, 2019 (JP) 2019-018249

(51) Int. Cl. A61B 3/00

(52) U.S. Cl. CPC A61B 3/0083; A61B 3/0075; A61B 3/0016

(57) ABSTRACT

An ophthalmologic apparatus that inspects a subject eye of a subject. The ophthalmologic apparatus includes an optometry device for inspecting the subject eye, a base for supporting the optometry device, an face supporter for holding the subject eye at an optometry position by supporting a face of the subject, and a changing device for relatively changing a positional relationship between the optometry position and the base in a working distance direction of the optometry device.

### CROSS-REFERENCE TO RELATED APPLICATION

This application claims priority from Japanese Patent Application No. 2019-018248 and No. 2019-018249 filed Feb. 4, 2019, the entire subject-matter of which is incorporated herein by reference.

### TECHNICAL FIELD

The present disclosure relates to an ophthalmologic apparatus for inspecting a subject eye.

### BACKGROUND

As ophthalmologic apparatuses in the related art, for example, a scanning laser ophthalmoscope (SLO), an optical coherence tomography (OCT), a fundus camera, an eye refraction power measuring apparatus, a corneal shape measuring apparatus, a corneal endothelial cell imaging apparatus, a corner imaging apparatus, a tonometry apparatus, or a combination of above apparatuses are known. For example, the above-described apparatuses project light emitted from a light source through an inspection window formed in a housing onto a subject eye, and perform imaging or measurement of the subject eye based on the reflected light of the projected light.

In the ophthalmologic apparatus described above, by mounting an optical adapter suitable for inspection purposes to the inspection window, it is possible to change a focal position according to the imaging portion (for example, from a fundus to an anterior segment), to change an imaging magnification and an imagine angle of view, and a Projection of a target for corneal topography (for example, refer to JP-A-2009-11381.

Incidentally, when the optical adapter is mounted, for example, a protrusion of the optometry device front a surface of the housing at the subject side becomes larger as much as an amount of size of the optical adapter than that before the mounting. In this case, in order to secure a mounting space and an appropriate Working distance of the optical adapter, it is necessary to move a main body of the apparatus away from the subject eye in the working distance direction compared to the case of non-mounting. However, if the positional relationship between the subject eye and the main body of the apparatus is changed by moving, the optometry device, in addition to a movable range under an assumption of normal alignment, a movable range needs to be further provided under an assumption of removal and attachment of the optical adapter. Therefore, the size of the apparatus increases.

### SUMMARY

In view of the problems in the related art, the present disclosure has an object to provide an ophthalmologic apparatus that can perform the inspection at an appropriate working distance while suppressing the increase in size of the apparatus.

In order to solve the problems described above, the present disclosure includes following configurations.

(1) There may be provided an ophthalmologic apparatus that inspects a subject eye of a subject including: an optometry device that inspects the subject eye; a base that supports the optometry device; a face supporter that holds the subject eye at an optometry position by supporting a face of the subject; and a changing device that relatively changes a positional relationship between the optometry position and the base in a working distance direction of the optometry device.

(2) In the ophthalmologic apparatus according to the above (1), the changing device relatively moves the base and the face supporter in the working distance direction to change the positional relationship.

(3) In the ophthalmologic apparatus according to the above (2), the changing device moves the face supporter with respect to the base.

(4) In the ophthalmologic apparatus according to the above (2), the changing device moves the base with respect to the face supporter.

(5) In the ophthalmologic apparatus according to the above (1), the face supporter is a forehead pad or a chin rest.

(6) In the ophthalmologic apparatus according to the above (1), further including: a controller configured to notify an inspector that the optometry position should be changed by the changing device.

(7) In the ophthalmologic, apparatus according to the above (1), the changing device includes a driver, and drives the driver to relatively move the base and the face supporter.

(8) In the ophthalmologic apparatus according to the above (7), further including: a change detector that detects whether or not the optometry position is changed by the changing device; and a controller configured to notify an inspector that the optometry position should be changed by the changing device or to control the driver based on a result of detection by the change detector.

(9) In the ophthalmologic apparatus according to the above (7), further including: an adapter detector that detects whether or not an optical adapter is mounted on the optometry device; and a controller configured to notify an inspector that the optometry position should be changed by the changing device or to control the driver based on a result of detection by the adapter detector.

(10) In the ophthalmologic apparatus according to the above (9), the adapter detector detects a type of the optical adapter, and the controller notifies the inspector that the optometry position should be changed by the changing device or controls the driver according to the type of the optical adapter.

(11) In the ophthalmologic apparatus according to the above (2), the changing device includes a movement locking device that locks the relative movement of the base and the face supporter to change the optometry position to a predetermined position corresponding to an optical adapter mounted on the optometry device.

(12) In the ophthalmologic apparatus according to the above (1), the changing device changes the optometry position by a distance corresponding to an optical adapter mounted on the optometry device.

(13) In the ophthalmologic apparatus according to the above (1), the changing device changes the optometry position by equal to or more than 100 mm.

(14) In the ophthalmologic apparatus according to the above (1), the changing device includes a face support attachment that is attachable to the face supporter, and changes the positional relationship by mounting the face support attachment on the face supporter.

(15) In the ophthalmologic apparatus according to the above (1), further including: a rotator that horizontally rotates the face supporter with respect to the base.

(16) In the ophthalmologic apparatus according to the above (15), the rotator includes a rotation driver, and drives the rotation driver to rotate the face supporter.

(17) In the ophthalmologic apparatus according to the above (16), further including: a rotation detector that detects whether or not the face supporter is rotated by the rotator; and a controller configured to notify an inspector that the optometry position should be changed by the changing device or to control the rotation driver based on a result of detection by the rotation detector.

(18) In the ophthalmologic apparatus according to the above (15), the rotator includes a rotation locking device that locks the rotation of the face supporter when the face supporter is rotated to a predetermined angle corresponding to an optical adapter mounted on the optometry device.

(19) In the ophthalmologic apparatus according to the above (15), the rotator rotates the face supporter in a range of 10′ to 60′ to left and right respectively from a normal imaging direction.

(20) In the ophthalmologic apparatus according to the above (15), the rotator rotates a face support attachment that is attachable to the face supporter.

According to the present disclosure, it is possible to prevent the optometry device from contacting the subject. While preventing the increase in size of the apparatus.

### BRIEF DESCRIPTION OF DRAWINGS

FIG. 1 is a schematic diagram illustrating an appearance of an apparatus.

FIG. 2 is a diagram illustrating an internal configuration of an optometry device.

FIG. 3 is a diagram illustrating a schematic configuration of a changing device.

FIG. 4 is a diagram illustrating a movement of the changing device.

FIG. 5 is a diagram illustrating a modification example of the changing device.

FIG. 6 is a diagram illustrating a modification example of the changing device.

FIG. 7 is a diagram illustrating a modification example of the changing device.

FIG. 8 is a diagram illustrating a schematic configuration of a rotator.

FIG. 9 is a diagram illustrating a movement of the rotator.

FIG. 10 is a diagram illustrating a movement of the rotator.

FIG. 11 is a diagram illustrating a modification example of the rotator.

### DETAILED DESCRIPTION

### First Illustrative Embodiment

A first illustrative embodiment in the present disclosure will be described with reference to the drawings. An ophthalmologic apparatus in the first illustrative embodiment inspects a subject eye. The ophthalmologic apparatus includes, for example, an eye refraction power measuring apparatus, a corner measuring apparatus, a conical endothelial cell imaging apparatus, a tonometry apparatus, an ocular axial length measuring apparatus, a fundus camera, an optical coherence tomography (OCT), a scanning laser ophthalmoscope (SLO), and the like.

### Appearance of Apparatus

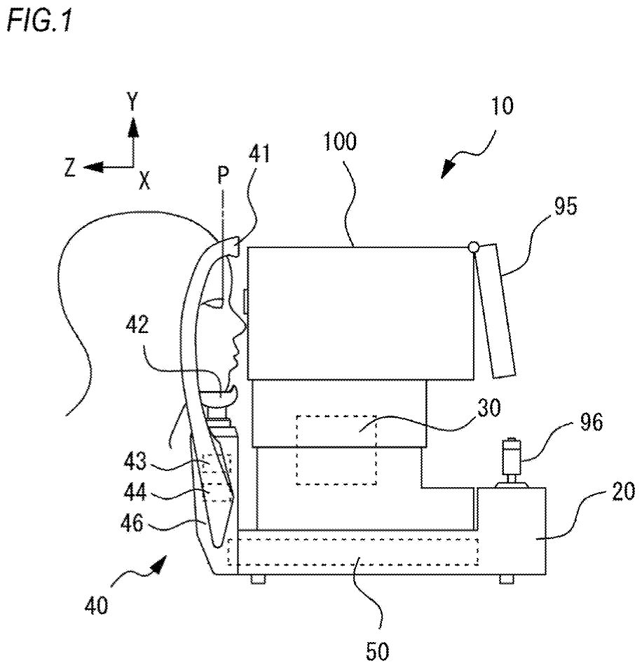

An appearance of the ophthalmologic apparatus will be described with reference to FIG. 1. As illustrated in FIG. 1, an ophthalmologic apparatus 10 in the present illustrative embodiment includes a base 20, an XYZ driving portion 30, a face supporter 40, a changing device 50, a display portion 95, an operation portion 96, and an optometry device 100.

The base 20 supports the entire apparatus. For example, the XYZ driving portion 30 moves the optometry device 100 in the up and down, left and right, and front and rear direction (three-dimensional direction) with respect to the base 20.

The face supporter 40 is, for example, a chin support. The face supporter 40 includes, for example, a forehead pad 41, a chin rest 42, a base portion 46, and the like. A forehead of the subject is in contact with the forehead pad 41. The chin rest 42 supports the chin of the subject. The chin rest 42 may be moved in the up and down direction according to the driving by a chin support driving portion 43. The base portion 46 supports the forehead pad 41 and the chin rest 42. The face supporter 40 may include a chin support sensor 44 that detects whether or not the chin is placed on the chin rest 42. For example, the chin support sensor 44 detects, for example, that the chin rest 42 has been pushed downward by the subject chin. The chin support sensor 44 may be, for example, a photo sensor, a magnetic sensor, a pressure sensor, a contact sensor, or the like.

The changing device 50 relatively changes a positional relationship between an optometry position P of the subject eye and the base 20 in the Z direction (the working distance direction of the optometry device 100). Details of the changing device 50 will be described later.

The display portion 95 displays, for example, the captured image of the subject eye or the result of measurement. For example, the display portion 95 may be provided integrally with the ophthalmologic apparatus 10 or may be provided separately from the ophthalmologic apparatus 10 and connected by wire or wirelessly. The display portion 95 may be a touch panel.

Various operation instructions by the inspector are input to the operation portion 96. The operation portion 96 outputs a signal corresponding to the input operation instruction. For the operation portion 96, for example, at least any one of user interfaces such as a mouse, a joystick, a keyboard, a trackball, a button and a touch panel may be used. When the display portion 95 is a touch panel, the display portion 95 may function as the operation portion 96.

### Optometry Device

The optometry device 100 inspects the subject eye. The optometry device 100 may include, for example, an optical system that measures an eye refraction power, a corneal curvature, an intraocular pressure, or the like of the subject eye. The optometry device 100 may include an optical system for imaging the anterior segment of the subject eye, the fundus or the like. In the present illustrative embodiment, as an example, a case where an optical system that captures a tomographic image of the subject eye is included is described.

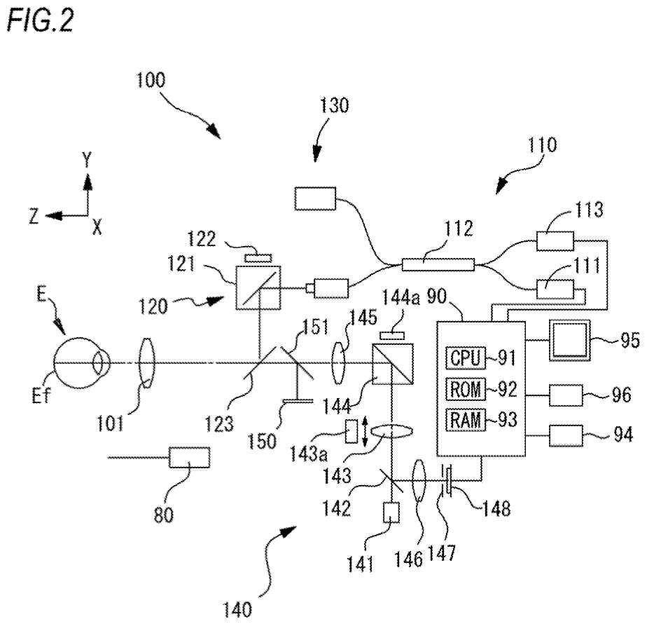

FIG. 2 is a schematic diagram illustrating an internal configuration of the optometry device 100. As illustrated in FIG. 2, the optometry device 100 in the present illustrative embodiment includes an OCT optical system 110, an observation optical system 140, a fixation target projection unit 150, a controller 90, and the like.

For example, the OCT optical system 110 irradiates the subject eye E with measurement light, and obtains an OCT signal obtained from the reflected light and the measurement light. For example, the OCT optical system 110 captures a tomographic image of the subject eye E by obtaining the OCT signal.

The OCT optical system 110 is an optical system of a so-called optical coherence tomography (OCT). The OCT optical system 110 divides the light emitted from a measurement light source 111 into measurement light (sample light) and reference light by a coupler (light splitter) 112. Then, the OCT optical system 110 guides the measurement light to a fundus Ef of the eye E by the measurement optical system 120. The measurement optical system 120 includes a scanning unit (for example, an optical scanner) 121, for example. For example, the scanning unit 121 changes the scanning position of the measurement light on the subject eye in order to change the imaging position on the subject eye. The OCT optical system 110 guides the reference light to a reference optical system 130. Thereafter, a detector 113 receives interference light generated by combining the measurement light reflected by the subject eye E and the reference light.

The detector 113 detects a state of interference between the measurement light and the reference light. In a case of Fourier domain OCT, a spectrum intensity of the interference light is detected by the detector 113, and a depth profile (A scan signal) in a predetermined range is obtained by Fourier transform on the spectrum intensity data. Examples include a spectrum-domain OCT (SD-OCT) and a swept-source OCT (SS-OCT). Alternatively, a time-domain OCT (TD-OCT) may be used.

The light emitted from the light source 111 is divided into a measurement light flux and a reference light flux by the coupler 112. The measurement light flux is emitted into the air after passing through the optical fiber. The light flux is condensed on fundus Ef through an optical member of the measurement optical system 120. Then, the light reflected by the fundus Ef is returned to the optical fiber through a similar light path.

The scanning unit 121 scans the measurement light in the XY direction (transverse direction) on the fundus. The scanning unit 121 is disposed at a position substantially conjugate with a pupil. For example, the scanning unit 121 is a galvano-scanner having two galvano-mirrors, and the reflection angle thereof is adjusted by a driving mechanism 122 at any way. The scanning unit 114 may be configured to deflect the light. For example, in addition to the reflective mirrors (galvano-mirror, polygon mirror, resonant scanner), an acousto-optic device (AOM) that changes the traveling (deflection) direction of the light is used.

The reference optical system 30 generates the reference light that is combined with the reflected light obtained by the reflection of the measurement light at the fundus Ef. The reference optical system 130 may be a Michelson type or a Mach-Zehnder type. The reference optical system 130 is formed by, for example, a reflection optical system (for example, a reference mirror), and returns the light from the coupler 112 back to the coupler 112 again through the reflection by the reflection optical system and guides the light to the detector 113. As another example, the reference optical system 130 is formed by a transmission optical system (for example, an optical fiber), and guides the light from the coupler 112 to the detector 113 by transmitting the light without returning.

The reference optical system 130 is configured to change an optical path length difference between the measurement light and the reference light by moving the optical member in the reference optical path. For example, the reference mirror is moved in the optical axis direction. The configuration for changing the optical path length difference may be disposed in the measurement light path of the measurement optical system 120.

The observation optical system 140 captures an observation image of the subject eye. The observation image may be, for example, a front image of the fundus Ef or an anterior segment image. The observation optical system 140 in the present illustrative embodiment is a so-called scanning laser ophthalmoscope (SLO). For example, the observation optical system 140 includes, for example, an light source 141, a focusing lens 143 a scanning unit 144, a relay lens 145, and the like. The SLO hula source 141 is a light source that emits highly coherent light, and for example, a laser diode light source with λ=780 nm is used. The focusing lens 143 is movable in the optical axis direction according to the refraction error of the subject eye. The scanning unit 144 includes a combination of a galvano-mirror and a polygon mirror that can scan the measurement light in the XY directions on the fundus at a high speed by driving of the drive unit 144a. The relay lens 145 relays the measurement light reflected by the scanning unit 144 to an objective lens 101.

A be splitter 142 is disposed between the SLO light source 141 and the focusing lens 143. In the reflection direction of the beam splitter 142, a condensing lens 146, a confocal aperture 147 placed at a position conjugate to the fundus, and a light receiving element 148 are provided.

The laser light (measurement light) emitted from the SLO light source 141 transmits the beam splitter 142 and then reaches the scanning unit 144 via the focusing lens 143, and the reflection direction is changed by driving of the galvano-mirror, the polygon mirror, or the like. Then, the laser light reflected by the scanning unit 144 is condensed on the fundus via the relay lens 145 and the objective lens 101.

The laser light reflected at the fundus passes through the objective lens 101, the relay lens 145, the scanning unit 144, and the focusing lens 143, and is reflected at the beam splitter 142. Thereafter, the light is condensed by the condensing lens 146, and then, detected by the light receiving element 148 through the confocal aperture 147. The light reception signal detected by the light receiving element 148 is input to the controller 90 described later. The controller 90 obtains the front image of the fundus of the subject eye based on the light reception signal obtained by the light receiving element 148. The obtained front image is stored in a storage unit 94. The obtainment of the SLO image is performed by vertical scanning (sub-scanning) of the laser light by the galvano-mirror provided in the scanning unit 144 and horizontal scanning (main scanning) of the laser light by the polygon mirror.

The configuration of the observation optical system 140 may be a so-called fundus camera type configuration. In addition, the OCT optical system 110 may be used as the observation optical system 140. That is, the front image may be obtained using data forming the tomographic image obtained two-dimensionally (for example, an integrated image in the depth direction of a three-dimensional tomographic image, an integrated value of the spectrum data at each XV position, or the like).

The fixation target projection unit 150 includes an optical system for guiding the viewing direction of the eye E. For example, the fixation target projection unit 150 presents a fixation target on the eye E. The fixation target projection unit 150 includes, for example, a visible light source that emits visible light. The fixation target projection unit 150 may be an internal fixation lamp type or an external fixation lamp type.

For example, the face imaging unit 80 images a face of the subject eye. For example, the face imaging unit 80 images a face including at least one of left and right subject eyes.

The controller 90 is realized by a general central processing unit (CPU) 91, ROM 92, RAM 93, and the like. The ROM 92 of the controller 90 stores an OCT signal processing program for processing the OCT signal, various programs for controlling the operation of the ophthalmologic apparatus 10, initial values, and the like. The RAM 93 temporarily stores various information. The controller 90 may be configured with a plurality of controllers 90 (that is, a plurality of processors).

As illustrated in FIG. 2, for example, a storage unit (for example, a non-volatile memory) 94, a display portion 95, an operation portion 96, and the like are electrically connected to the controller 90. The storage unit 94 is a non-transitory storage medium that can retain the stored contents even when the power supply is interrupted. For example, a hard disk drive, a flash ROM, a removable USB memory, or the like can be used as the storage unit 94.

### Changing Device

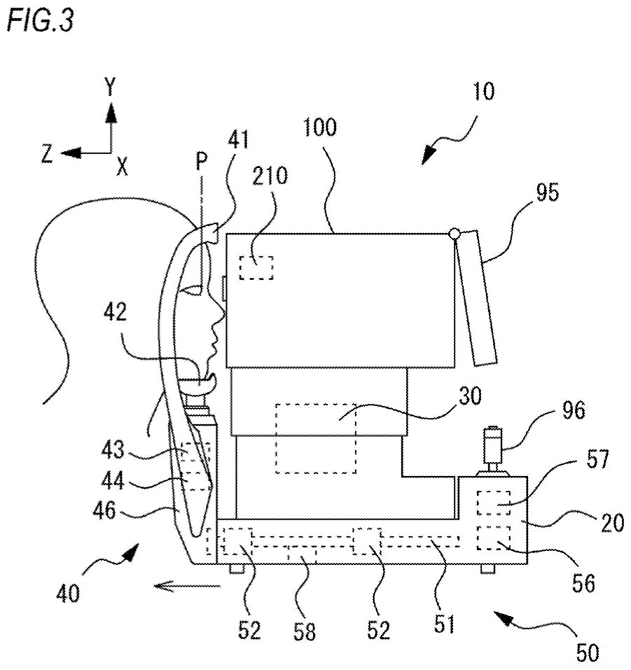

The changing device 50 in the present illustrative embodiment will be described with reference to FIG. 3. The changing device 50 in the present illustrative embodiment changes the optometry position P of the subject eye by moving the face supporter 40 in the front-rear direction (Z direction). As illustrated in FIG. 3, the changing device 50 includes, for example, a slide part 51 and a guide part 52. The slide part 51 slides (sliding) in the Z direction. The slide part 51 is, for example, a shaft or a plate member. The guide part 52 retains the slide part 51 so as to be slidable in the Z direction. The guide part 52 is, for example, a bush or a guide rail.

For example, one end of the slide part 51 is fixed to the face supporter 40 (for example, the base portion 46), and the other end side is inserted into the hole of the guide part 52. The guide part 52 is fixed to the base 20. Therefore, when the slide part 51 is inserted in the guide part 52 and is slid in the Z direction, the position of the face supporter 40 is changed with respect to the base 20.

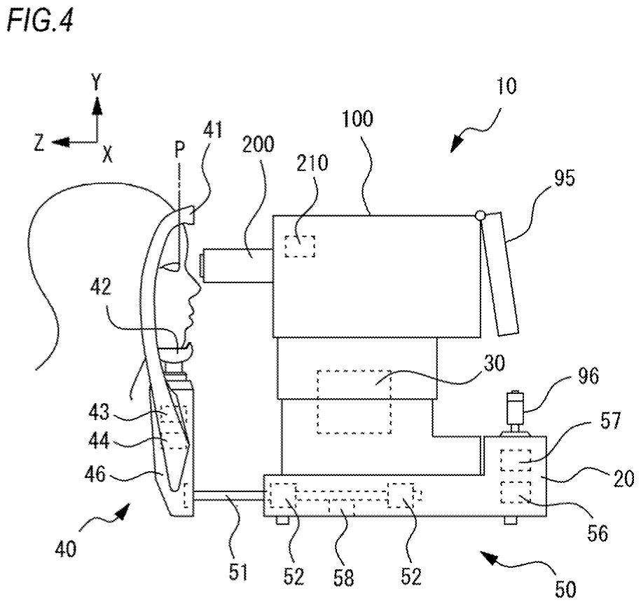

FIG. 4 illustrates a state when the optical adapter 200 is mounted on the optometry device 100. The optical adapter 200 is mounted on the inspection window of the optometry device 100 when performing unusual imaging such as wide-angle imaging or anterior segment imaging. In the optical adapter 200, optical elements necessary for performing various imaging are arranged. As illustrated in FIG. 4, when the optical adapter 200 is mounted on the optometry device 100, the inspector can move the optometry position P of the subject eye to a position corresponding to the optical adapter 200 by pulling out the face supporter 40 with respect to the base 20.

### Operation Procedure

An operation procedure of the ophthalmologic apparatus 10 including the above configuration will be described. First, a case of the normal imaging without mounting the optical adapter 200 will be described. The inspector instructs the subject to place the chin on the chin rest 42 and to contact the forehead to the forehead pad 41. When the chin of the subject is placed on the chin rest 42, a signal is sent from the chin support sensor 44 to the controller 90. When the signal from the chin support sensor 44 is received, the controller 90 starts the inspection by the optometry device 100. The controller 90 calculates the direction of the subject eye from the image captured by the face imaging unit 80, and moves the optometry device 100 in that direction. Thereafter, when the observation image of the subject eye can be captured by the observation optical system 140, alignment is automatically performed based on the captured observation image, and the inspection by the optometry device 100 is performed.

Next, a case where the wide-angle imaging is performed by mounting the optical adapter 200 on the optometry device 100 will be described. The inspector pulls out the face supporter 40 from the base 20 and secures a space between the optometry device 100 and the face supporter 40. The inspector mounts the optical adapter 200 on the optometry device 100. Similarly to the normal imaging, when the chin of the subject is placed on the chin rest 42 according to the instruction by the inspector, the controller 90 starts the inspection of the subject eye. The controller 90 calculates the direction of the subject eye from the image captured by the face imaging unit 80, and moves the optometry device 100 in that direction. Thereafter, when the observation image of the subject eye can be captured by the observation optical system 140, alignment is automatically performed based on the captured observation image, and the inspection by the optometry device 100 is performed.

As described above, by moving the face supporter 40 in the Z direction, the optometry position P of the subject eye can be changed with respect to the base 20. In this way, it is possible to prevent the optical adapter 200 from contacting the nose of the subject, and the subject eye can be inspected at an appropriate working distance to the optical adapter 200. In addition, the optical adapter 200 can be prevented from coming into contact with the face supporter 40 or the like when mounting the optical adapter 200. Furthermore, it is not necessary to increase the movable range of the optometry device 100 in the Z direction in consideration of the length of the optical adapter 200, and the expansion range can be reduced even if the movable range is increased. In addition, when the optical adapter 200 is used, it is possible to save the efforts of greatly moving the optometry device 100 in the Z direction.

### Modification Example

In the first illustrative embodiment, the changing device 50 includes the slide part 51 and the guide part 52, but other moving mechanisms may be used. For example, as the changing device 50, a link mechanism, a telescopic mechanism, or the like may be used.

In the first illustrative embodiment, the changing device 50 moves the entire face supporter 40, but a part of the face supporter 40 may he moved. For example, the changing device 50 may relatively change the optometry position P of the subject eye with respect to the base 20 by moving the forehead pad 41 or the chin rest 42 in the Z direction with respect to the base 20.

In the first illustrative embodiment, the changing device 50 moves the face supporter 40 with respect to the base 20, but the base 20 may be moved with respect to the face supporter 40. That is, the position of the base 20 may be changed while the position of the face supporter 40 is fixed. In this case, for example, leg portions may be provided on the face supporter 40 and fixed to an optical table 300, and a movement mechanism such as a wheel may be provided on the base 20 so as to move on the optical table 300.

For example, when it is necessary to change the optometry position P such as a case when an imaging mode is switched to a mode of using the optical adapter 200, the controller may have a function of notifying the inspector of the change. For example, the controller 90 may control the display portion 95 to display an instruction to pull out the face supporter 40 from the base 20 on the display portion 95 in order to notify the inspector of the chance. In addition, the controller 90 may control a speaker or the like to generate a sound for urging the inspector to switch the optometry position P.

The changing device 50 may include a Z drive unit 56. For example, the Z drive unit 56 automatically moves the slide part 51 in the Z direction. The inspector may operate the driving of the Z drive unit 56 using the operation portion 96. In this way, the inspector can automatically pull out the face supporter 40 without manually pulling out the face supporter 40.

The ophthalmologic apparatus 10 may include a change detector 57 (refer to FIG. 3). The change detector 57 detects whether or not the optometry position P is changed by the changing device 50. The change detector 57 may detect the position of the slide part 51 using, for example, an encoder, a micro switch, a position sensor, or the like. If the change detector 57 is provided, the controller 90 may control the ophthalmologic apparatus 10 according to the result of detection by the change detector 57. For example when the change detector 57 detects that the face supporter 40 is not moved from the normal imaging position even though the imaging mode is set as the mode of using the optical adapter 200, the controller 90 may drive the Z drive unit 56 to move the face supporter 40 to a position of wide-angle imaging or anterior segment imaging, or may display an instruction to pull out the face supporter 40 from the base 20 on the display portion 95 or the like.

The opthalmologic apparatus 10 may include an adapter detector 210. For example, the adapter detector 210 detects whether or not the optical adapter 200 is mounted on the optometry device 100. For example, the adapter detector 210 may detect the presence or absence of the optical adapter 200 using an optical sensor, or the like. When the adapter detector 210 is provided, the controller 90 may control the ophthalmologic apparatus 10 according to the presence or absence of the optical adapter 200. For example, when it is detected that the optical adapter 200 is mounted, the controller 90 may drive the Z drive unit 56 to move the face supporter 40 to a position of wide-angle imaging or anterior segment imaging, or may display the instruction to pull out the face supporter 40 from the base 20 on the display portion 95 or the like.

The adapter detector 210 may detect the type of the optical adapter 200. In this case, the controller 90 may control the ophthalmologic apparatus 10 according to the type of the optical adapter 200. For example, if the adapter detector 210 detects that a wide-angle adapter used for wide-angle imaging is mounted, the controller 90 may drive the Z drive unit 56 to move the face supporter 40 to the position of wide-angle imaging, or may display the instruction to move the face supporter 40 to the position of wide-angle imaging on display portion 95 or the like. Similarly, if the adapter detector 210 detects that an anterior segment adapter used for anterior segment imaging is mounted, the controller 90 may drive the Z drive unit 56 to move the face supporter 40 to the position of anterior segment imaging, or may display the instruction to move the face supporter 40 to the position of anterior segment imaging on the display portion 95 or the like.

The changing device 50 may be able to change the optometry position P to a predetermined position corresponding to the optical adapter 200. For example, the changing device 50 may include a movement locking portion 58. The movement locking portion 58 may lock the movement of the slide part 51 when the slide part 51 moves as much as a predetermined distance. In this way, it becomes easy to dispose the optometry position P at an appropriate position according to the optical adapter 200. In addition, if there are a plurality of types of optical adapters 200 mounted on the optometry device 100, a plurality of movement locking portions 58 corresponding to each optical adapter 200 may be provided such that the slide part 51 is locked at a plurality of predetermined positions.

For example, the movement locking portion 58 may be a click mechanism. For example, the click mechanism is a mechanism that provides a click feeling for temporal fixing h dropping a ball biased by a spring into a groove provided at a predetermined position. In the temporarily fixed state, by applying a force of a certain level or more, the ball is removed from the groove and the temporal fixing is released. Therefore, a groove may be provided such that the ball falls when the slide part 51 is disposed at a predetermined position.

The changing device 50 may be configured to change the optometry position P by equal to or more than 100 mm in consideration of the length of the optical adapter 200. More preferably, the optometry position P may be changed equal to or more than 150 mm. As a result, the more types of optical adapter 200 can be supported. Of course, the changing device 50 may be configured to change the optometry position P within a range of less than 100 mm in order to support the short optical adapter 200.

The changing device 50 may be configured integrally with the face supporter 40 or the base 20. That is, the face supporter 40 or the base 20 may include the changing device 50.

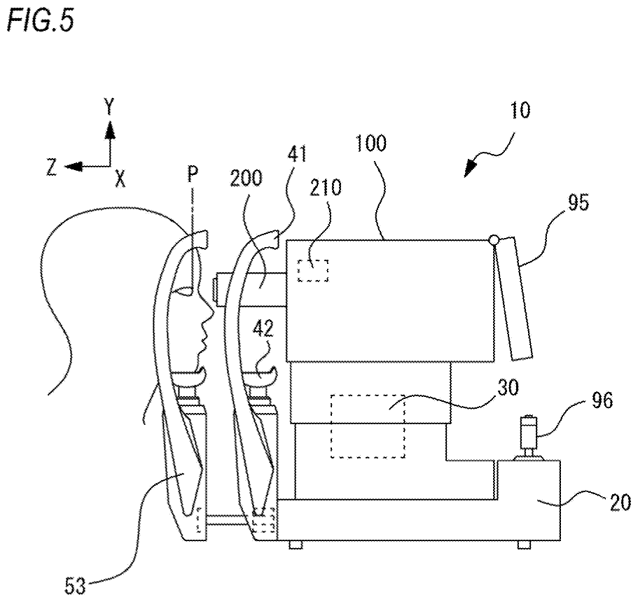

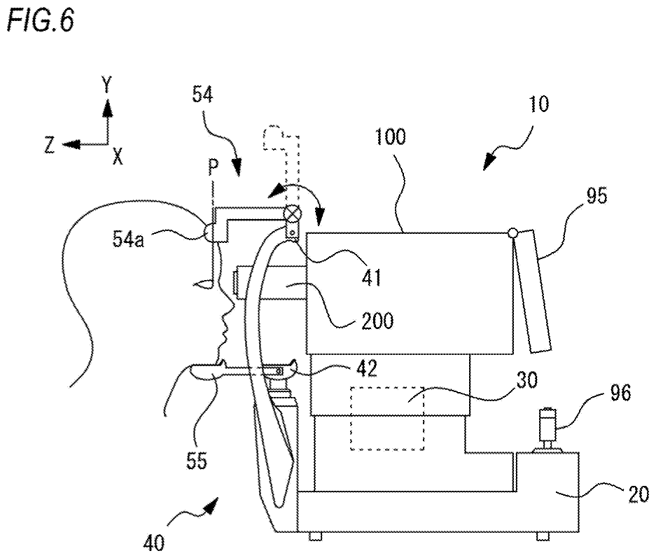

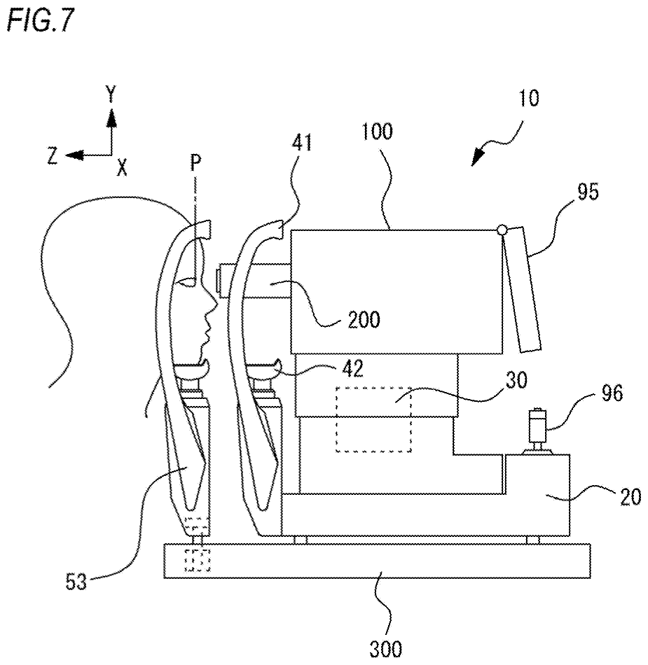

In the first illustrative embodiment, the changing device 50 changes the optometry position P by moving the face supporter 40, but not limited thereto. For example, the changing device 50 may change the optometry position P by mounting an attachment on the face supporter 40 or the base 20. For example, the changing device 50 may include a face support attachment 55, a forehead pad attachment 54, or a chin rest attachment 55 as the attachment to be mounted on the face supporter 40 or the base 20 (refer to FIG. 5 and FIG. 6). In this way, the optometry position P with respect to the base 20 can be relatively changed in the Z direction. As illustrated in FIG. 6, the forehead pad attachment 54 may include a forehead pad 53a provided to be rotatable in the vertical direction. In this way, when the optical adapter 200 is not mounted, the forehead pad 54a can be lifted up and retracted. The face support attachment 53 may be mounted on the optical table 300 not limited to the a body of the apparatus as illustrated in FIG. 7.

The optical adapter 200 mounted on the optometry device 100 may be used for measuring the subject eye, not limited to be used for imaging the subject eye. For example, the optical adapter 200 may be a ring cone for topography. For example, the ring cone projects a plurality of ring targets (placido targets) onto the cornea of the subject eye.

The ophthalmologic apparatus 10 may be provided with a fixing portion for fixing the base 20 and the optical table 300 such that the main body of the apparatus does not fall down when moving the face supporter 40 with respect to the base 20.

### Second Illustrative Embodiment

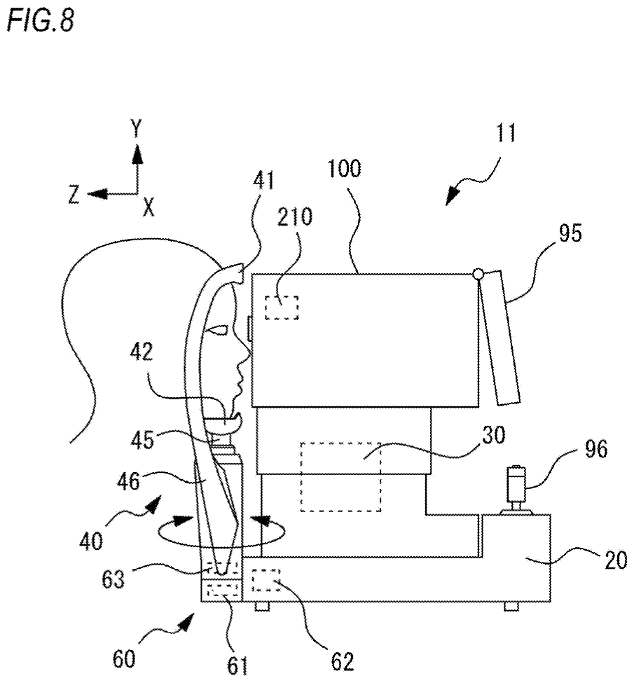

A second illustrative embodiment will be described. As illustrated in FIG. 8, an ophthalmologic imaging apparatus 11 in the second illustrative embodiment includes a rotator 60. The rotator 60 rotates the face supporter 40 in the horizontal direction, for example. Since other configurations are the same as those in the first illustrative embodiment, the same reference numerals are given and the descriptions thereof will not be repeated.

As illustrated in FIG. 8, for example, the rotator 60 is fixed to the base 20, and the face supporter 40 is fixed on the rotator 60. The rotator 60 includes, for example, a rotation mechanism such as a turntable to rotate the face supporter 40 in the horizontal direction with respect to the base 20. For example, the rotator 60 rotates the face supporter 40 around the central axis of the support column 45 that supports the chin rest 42. Of course, the rotator 60 may rotate the face supporter 40 in a circular or elliptical orbit.

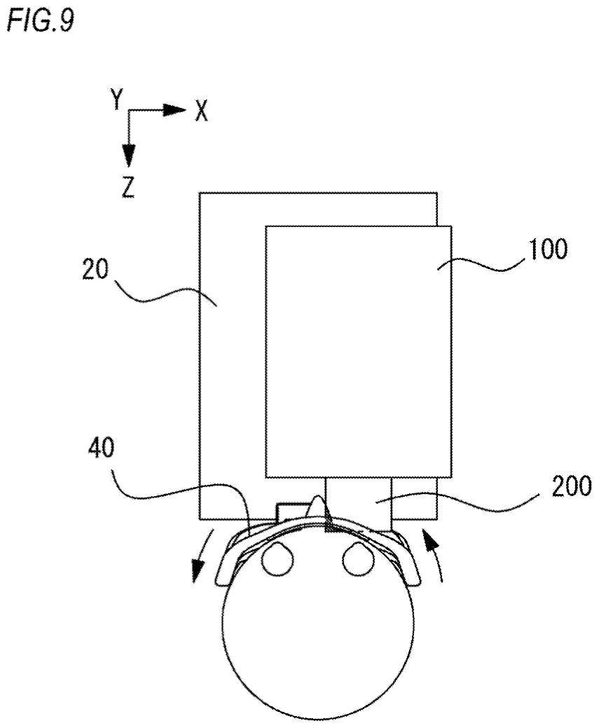

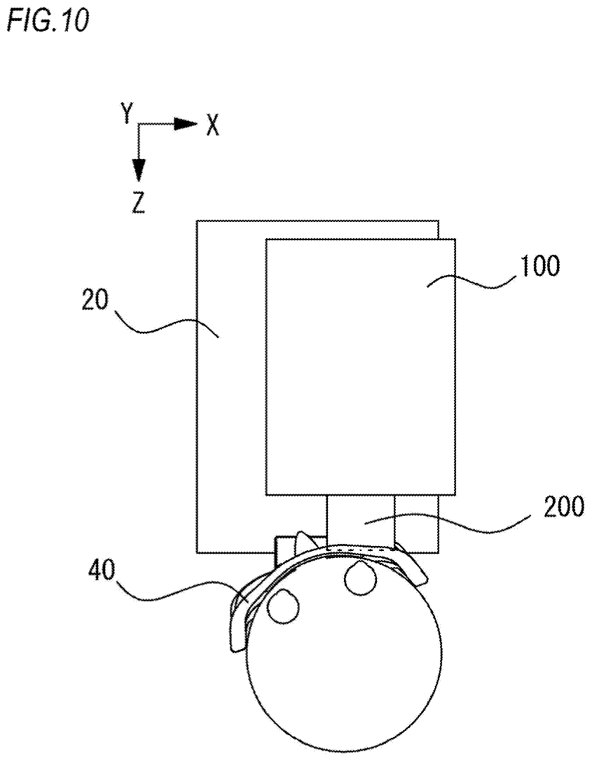

As illustrated in FIG. 9, when the optometry device 100 approaches the subject under the state that the optical adapter 200 is mounted OD the optometry device 100, the optometry device 100 may come into contact with the nose or the like. In such a case, as illustrated in FIG. 10, by rotating the face supporter 40 in the horizontal direction with respect to the base 20, and changing the orientation of the face of the subject, it is possible to perform imaging so that the optical adapter 200 does not come in contact with the nose of the subject. In addition, by rotating the face supporter 40 around the central axis of the support column 45 (the vertical movement axis of the chin rest 42), complication of the apparatus configuration can be prevented.

### Modification Example

In the second illustrative embodiment, the rotator 60 rotates the entire face supporter 40, but may rotate a part of the face supporter 40. For example, the rotator 60 may rotate the forehead pad 41 or the chin rest 42 in the horizontal direction with respect to the base 20. As described above, even when a part of the face supporter 40 is rotated in the horizontal direction, the orientation of the face of the subject can be changed. The rotator 60 may be provided on both the chin rest 42 and the forehead pad 41.

If the face supporter 40 needs to be rotated, the controller 90 may notify the inspector by displaying an instruction such as “please turn the face supporter 40” on the display portion 95 or the like, for example, when the imaging mode is switched to the mode of using the optical adapter 200. In addition, the face supporter 40 may be urged to rotate by generating to sound through a speaker or the like and notifying the inspector.

The rotator 60 may include a rotation driver 61 (refer to FIG. 8). For example, the rotation driver 61 automatically rotates the rotator 60 in the horizontal direction. For example, the inspector may operate the drive of the rotation driver 61 by the operation portion 96. Accordingly, the inspector can automatically rotate the face supporter 40, without manually rotating.

The ophthalmologic imaging apparatus 11 may include a rotation detection portion 62 (refer to FIG. 8). The rotation detection portion 62 detects whether or not the face supporter 40 is rotated by the rotator 60. The controller 90 may control the ophthalmologic imaging apparatus 11 according to the result of detection by the rotation detection portion 62. For example, when the rotation detection portion 62 detects that the face supporter 40 is not rotated from the normal imaging direction even though the imaging mode is set as the mode of using the optical adapter 200, the controller 90 may drive the rotation driver 61 to rotate the face supporter 40 in a direction that does not easily interfere with the nose. In addition, when the rotation detection portion 62 detects that the face supporter 40 is not rotated from the normal imaging direction even though the imaging mode is set as the mode of using the optical adapter 200, the controller 90 may display an instruction to rotate the face supporter 40 on the display portion 95 or the like.

When the controller 90 includes the adapter detector 210, the controller 90 may control the ophthalmologic imaging apparatus 11 according to the presence or absence of the optical adapter 200. For example, when the adapter detector 210 detects that the optical adapter 200 is mounted, the controller 90 may drive the rotation driver 61 to rotate the face supporter 40 to the angle of wide-angle imaging or anterior segment imaging, or may display an instruction to rotate the face supporter 40 on the display portion 95 or the like.

The rotator 60 may allow the face supporter 40 to rotate by a predetermined angle. For example, the rotator 60 may include a rotation locking device 63. The rotation locking device 63 may lock the rotation of the rotator 60 when the face supporter 40 rotates by a predetermined angle. In this way, the inspector can rotate the face supporter 40 to an angle suitable for mounting the optical adapter 200.

The rotation locking device 63 may be a click mechanism, for example. For example, a groove may be provided such that a ball of the click mechanism falls at a position where the face supporter 40 is rotated by a predetermined angle.

The rotator 60 may allow the face supporter 40 to rotate by a plurality of predetermined angles according to the type of the optical adapter 200 to be mounted on the optometry device 100. In this case, the rotation of the rotator 60 may be locked at a plurality of predetermined angles by the rotation locking device 63. For example, in a case where the rotation locking device 63 is a click mechanism, a plurality of click mechanism grooves may be disposed to have a plurality of predetermined angles.

The rotator 60 may be configured such that the face supporter 40 can be changed in a range of 10° to 60° in consideration of the shape of the optical adapter 200. More preferably, it can be changed at equal to or more than 20°. As a result, the more types of optical adapter 200 can be supported.

The rotator 60 may be configured as a part of the face supporter 40 or the base 20. That is, the face supporter 40 or the base 20 may include the rotator 60.

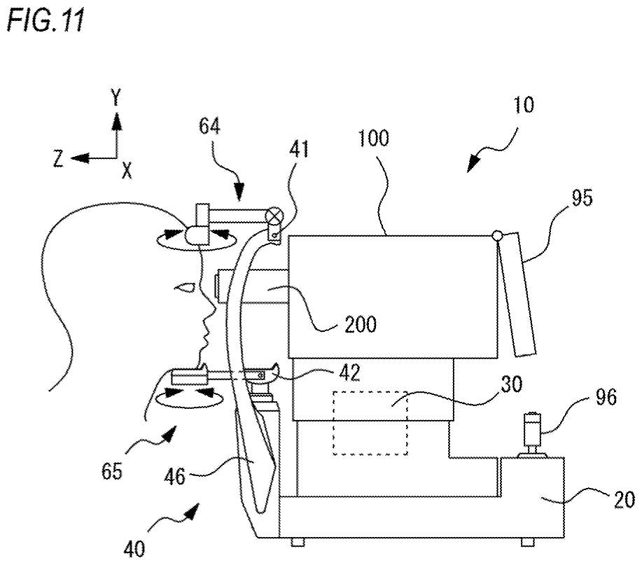

The rotator 60 may change the orientation of the face of the subject by rotating the attachment mounted on the face supporter 40 or the base 20 in the horizontal direction. For example, the rotator 60 may include a forehead pad attachment 64 or a chin rest attachment 65 as the attachment mounted on the face supporter 40 or the base 20 (see FIG. 11).

In the ophthalmologic apparatus 10 or the ophthalmologic imaging apparatus 11, both the changing device 50 and the rotator 60 may be provided. In this way, the inspector can move the face supporter 40 in the Z direction or can rotate the eye support portion 40 in the horizontal direction, and thus, it is possible to inspect the subject eye in an appropriate state without bringing the optical adapter 200 into contact with the nose of the subject.

The foregoing description of the illustrative embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the claims and their equivalents.

What is claimed is:

1. An ophthalmologic apparatus that inspects a subject eye of a subject, comprising:
an optometry device that inspects the subject eye;
a base that supports the optometry device;
a face supporter that holds the subject eye at an optometry position by supporting a face of the subject; and
a changing device that relatively changes a positional relationship between the optometry position and the base in a working distance direction of the optometry device.

2. The ophthalmologic apparatus according to claim 1,
wherein the changing device relatively moves the base and the face supporter in the working distance direction to change the positional relationship.

3. The ophthalmologic apparatus according to claim 2,
wherein the changing device moves the face supporter with respect to the base.

4. The ophthalmologic apparatus according to claim 2,
wherein the changing device moves the base with respect to the face supporter.

5. The ophthalmologic apparatus according to claim 1,
wherein the face supporter is a forehead pad or a chin rest.

6. The ophthalmologic apparatus according to claim 1, further comprising:
a controller configured to notify an inspector that the optometry should changed by the changing device.

7. The ophthalmologic apparatus according to claim 1,
wherein the changing device includes a driver, and drives the driver to relatively move the base and the face supporter.

8. The ophthalmologic apparatus according to claim 7, further comprising:
a change detector that detects whether or not the optometry position is changed by the changing device; and
a controller configured to notify an inspector that the optometry position should be changed by the changing device or to control the driver based on a result of detection by the change detector.

9. The ophthalmologic apparatus according to claim further comprising:
an adapter detector that detects whether or not an optical adapter mounted on the optometry device; and
a controller configured to notify an inspector that the optometry position should be changed by the changing device or to control the driver based on a result of detection by the adapter detector.

10. The ophthalmologic apparatus according to claim 9,
wherein the adapter detector detects a type of the optical adapter, and
wherein the controller notifies the inspector that the optometry position should be changed by the changing device or controls the driver according to the type of the optical adapter.

11. The ophthalmologic apparatus according to claim 2,
wherein the changing device includes a movement locking device that locks the relative movement of the base and the face supporter to change the optometry position to a predetermined position corresponding to an optical adapter mounted on the optometry device.

12. The ophthalmologic apparatus according to claim 1,
wherein the changing device changes the optometry position by a distance corresponding to an optical adapter mounted on the optometry device.

13. The ophthalmologic apparatus according to claim 1,
wherein the charging device changes the optometry position by equal to or more than 100 mm.

14. The ophthalmologic apparatus according to claim 1,
wherein the changing device includes a face support attachment that is attachable to the face supporter, and changes the positional relationship by mounting the face support attachment on the face supporter.

15. The ophthalmologic apparatus according to claim 1, further comprising:
a rotator that horizontally rotates the face supporter with respect to the base.

16. The ophthalmologic apparatus according to claim 15,
wherein the rotator includes a rotation driver, and drives the rotation driver to rotate the face supporter.

17. The ophthalmologic apparatus according to claim 16, further comprising:
a rotation detector that detects whether or not the face supporter is rotated by the rotator; and
a controller configured to notify an inspector that the optometry position should he changed by the changing, device or to control the rotation driver based on a result of detection by the rotation detector.

18. The ophthalmologic apparatus according to claim 15,
wherein the rotator includes a rotation locking device that locks the rotation of the face supporter when the face supporter is rotated to a predetermined angle corresponding to an optical adapter mounted on the optometry device.

19. The ophthalmologic apparatus according to claim 15,
wherein the rotator rotates the face supporter in a range of 10° to 60° to left and right respectively from a normal imaging direction.

20. The ophthalmologic apparatus according to claim 15,
wherein the rotator rotates a face support attachment that is attachable to the face supporter.
